(19) United States
(12) Patent Application Publication
(10) Pub. No.: US 2007/0134740 A1
(43) Pub. Date: Jun. 14, 2007

(54) Compositions and articles for detection of analytes exceeding a pre-set threshold

(76) Inventors: David Brusilovsky, Herzliya (IL); Menashe Terem, Yavne (IL); Adva Brand, Benyamina (IL)

Correspondence Address: WINSTON & STRAWN LLP;PATENT DEPARTMENT, 1700 K STREET, N.W., WASHINGTON, DC, 20006, US

(21) Appl. No.: 11/541,198

(22) Filed: Sep. 28, 2006

Related U.S. Application Data
(60) Provisional application No. 60/749,043, filed on Dec. 12, 2005.
(60) Provisional application No. 60/778,840, filed on Mar. 6, 2006.

(51) Int. Cl. G01N 33/569; G01N 33/554; G01N 33/558

(52) U.S. Cl. 435/7.22; 436/514; 435/7.32

(57) ABSTRACT

The present invention provides a bodily fluid-testing composition for the determination and quantification of a specific ion concentration exceeding a pre-set threshold in a tested fluid, in which an ion oppositely charged to the ion in the bodily fluid is used to compete with an indicator reagent in order to compensate for variability in specific binding of the bodily fluid ions. The present invention further provides an article for monitoring of bodily fluids comprising a substrate and an absorbent material for absorbing the bodily fluid. The substrate includes a composition suitable for identification of a specific ion concentration in a tested fluid. The article can be used to indicate the presence of abnormal ammonium concentration in human urine, amniotic fluid leakage, or biogenic secretions associated with bacterial vaginosis, parasite infections, or deficiency of lactobacillus population, without giving a false positive result.

This application claims the benefit of application Nos. 60/749,043 filed Dec. 12, 2005 and 60/778,840 filed Mar. 6, 2006. The entire content of each provisional application is expressly incorporated herein by reference thereto.

### FIELD OF THE INVENTION

The present invention relates to the field of medical diagnostics and more specifically, to improved identification of bodily fluids by using a bodily fluid-testing compositions and articles comprising same for determining the presence or concentration of analytes of interest exceeding a pre-set threshold in bodily fluids.

### BACKGROUND OF THE INVENTION

Various analytical procedures and devices for diagnosing medical conditions are commonly employed in assays comprising means for determining the presence and/or concentration of analytes of interest in bodily fluids. U.S. Pat. Nos. 4,266,022; 5,217,444; 5,445,147; 5,468,236; 5,660,790; 5,823,953; 5,910,447; 6,099,801; 6,106,461; 6,126,597; 6,149,590; 6,203,496; 6,562,297 and 6,689,114 disclose compositions with chemically reactive means adapted to provide a visual indication as a result of interacting with biological fluids, and disposable absorbent products comprising same. The visual indication provided by these absorbent products is not adapted to distinguish between indication for a substance of interest and the indication from interfering fluids, particularly, urine. Moreover, some of the articles disclosed and claimed in the aforementioned patents are directed to indicate the presence or absence of specific anlaytes in urine, e.g. for the purpose of determining dehydration as disclosed in U.S. Pat. No. 6,203,496, and thus cannot be applied for determining medical conditions in vaginal secretions.

U.S. Pat. No. 5,897,834 discloses a device that is capable of differentiating between urine and vaginal secretions associated with vaginosis or amniotic fluid. The device includes indicators with a negatively charged group immobilized to a solid polymer substrate containing quaternary ammonium groups. The polymer substrate however is ineffective in non-clinical settings as the indication from pH of dried vaginal secretions is vague and often invisible.

European Patent No. 586 590 discloses binding assays for determining the presence or amount of an analyte of interest in a test sample, using a binding pair, such as an antibody and antigen, a capture reagent comprising the first member of the binding pair, an indicator containing the second member of the binding pair and a detectable label and a solid phase material containing a polymeric cation reaction site. The assays of EP 586 590 are limited to immunoassay formats.

WO 2005/093414 discloses an assay device for detecting the presence or absence of amines within a test sample comprising a fluidic medium that defines a detection zone, wherein a chemichromic dye, specifically arylmethanes is contained within said detection zone, said chemichromic dye being capable of undergoing a detectable color change upon reaction with one or more amines.

U.S. Pat. Nos. 6,627,394 and 6,921,647, assigned to the applicant of the present invention, disclose a secretion-monitoring article for identifying a secreted biological fluid comprising a body with an absorbent material, at least one pH determining member and a reagent associated with the absorbent material. The article is capable of indicating the presence of amniotic fluid, or secretions associated with bacterial, parasite, fungal, or yeast infections without giving a false positive result upon exposure to urine. However, it is impracticable to obtain an indication that is above or below a pre-determined threshold level. Moreover, it takes a while for the indication (change in color) to occur, namely, up to about 20 minutes.

There is an unmet need for a bodily fluid-testing composition for the identification of a specific ion concentration exceeding a pre-set threshold in a tested bodily fluid, which can differentiate between a specific bodily fluid of interest and an interfering bodily fluid, while reducing the amount of time required obtaining the reliable result.

### SUMMARY OF THE INVENTION

The present invention provides compositions and articles comprising same capable of providing a visible indication of an analyte of interest in bodily-fluids, the concentration of which is above a predetermined threshold. The compositions of the invention comprise a pre-formed polymer, a plasticizer, a wetting agent, an indicator reagent, an ion-balance reagent and a competitive reagent having the same charge as the indicator reagent.

The compositions of the invention overcome the disadvantages of the prior art as they include a competing substance oppositely charged to the analyte of interest and having a binding affinity to said analyte stronger than the affinity of the indicator of the composition to said analyte. Without wishing to be bound by any particular theory or mechanism of action, the competing substance competes with an indicator reagent in order to compensate for variability in specific binding of the analyte with said indicator. Thus, the ability of the compositions of the invention to detect a specific ion concentration, above or below a pre-set threshold, is determined by the concentration of the competitor.

The present invention further provides method for detecting the presence and amount of analytes of interest in bodily fluids, comprising using the compositions and articles of the invention.

Surprisingly, the articles and compositions of the present invention produce highly specific and highly sensitive diagnostic indications, with minimal “noise” (interference) from nonspecific binding of interfering substances, and, thereby offering improved accuracy of analysis.

According to one aspect, the present invention provides a composition for determining the presence of a charged analyte of interest in a tested bodily fluid, comprising a pre-formed polymer, an indicator reagent being charged oppositely to an analyte of interest in a tested bodily fluid, a competitive reagent having the same charge as the indicator reagent, and an ion-balance reagent, wherein the binding affinity of the competitive reagent to the analyte is stronger than the binding affinity of the indicator reagent to said analyte, and wherein the concentration of the competitive reagent determines a pre-set threshold of a visible indication such that upon contact of the composition with a bodily fluid comprising said analyte in a concentration above the pre-set threshold, said composition changes color.

According to one embodiment,. According to an alternative embodiment, the indicator reagent and the competitive reagent are positively charged.

According to a preferred embodiment, the indicator reagent is a weak organic acid and the competitive reagent is an organic acid. According to an alternative embodiment, the indicator reagent is a weak organic base and the competitive reagent is an organic base.

According to another embodiment, the indicator reagent is selected from the group consisting of methyl yellow, methyl orange, bromophenol blue, alizarin sodium sulfonate, naphtyl red, bromcresol green, methyl red, bromcresol purple, nitrazine yellow, bromoxylenol blue, neutral red, phenol red, thymol blue, xylenol blue, m-cresol purple, naphtholthalein, phenolphthalein and naphtholbenzein.

According to yet another embodiment, the competitive reagent is selected from the group consisting of citric acid, oxalic acid, tartaric acid, succinic acid, glutaric acid, lactic acid, pyruvic acid, hydroxypropionic acid, hydroxyvaleric acid, adipic acid, suberic acid, orotic acid, phthalic acid, 2-[(2-hydroxy-1,1-bis(hydroxymethyl)ethyl)amino]ethanesulfonic acid and 4-(2-hydroxyethyl)piperazine-1-ethanesulfonic acid. According to a further embodiment, the competitive reagent is selected from the group consisting of cyclodextrine sulfate, dextran sulfate, and carboxymethyl cellulose.

According to yet another embodiment, the ion-balance reagent is a quaternary amine. According to yet another embodiment, the ion-balance reagent is selected from the group consisting of di(long-chain alkyl)dimethyl ammonium chloride, N-methyl-N,N-bis(long-chain alkanoyl oxyethyl)-N-(2-hydroxyethyl) ammonium methylsulfate, vinylbenzyl dimethylcocoammonium chloride, and methyl trioctyl ammonium chloride and tri-dodecylmethyl ammonium chloride.

According to yet another embodiment, the pre-formed polymer is selected from the group consisting of cellulose acetate, cellulose, sodium carboxymethyl cellulose, ethyl cellulose, and nitrocellulose.

According to yet another embodiment, the composition further comprises at least one compound selected from: a wetting agent and a plasticizer.

According to yet another embodiment, the wetting agent is selected from the group consisting of 2-ethoxy ethanol, triethylene glycol, ethylene glycol and sorbitol.

According to yet another embodiment, the plasticizer is selected from the group consisting of dibutylphthalate, dioctylphthalate, castor oil, diacetylated monoglycerides, diethyl phthalate, glycerin, mono- and di-acetylated monoglycerides, polyethylene glycol, propylene glycol, triacetin, triethyl citrate, bis-(2-butoxyethyl) adipate, and bis-(2-ethylhexyl) sebacate.

According to yet another embodiment, the pre-formed polymer is in an amount that does not exceed about 40%; the plasticizer is in an amount that does not exceed about 35%; the wetting agent is in an amount that does not exceed about 40%; the ion-balance reagent is in an amount that does not exceed about 30%; the competitive reagent is in an amount that does not exceed about 8% and the indicator reagent is in an amount that does not exceed about 2%; wherein the percents are weight percent based on the total dry weight of the composition and the total dry weight of the composition equals 100%.

According to yet another embodiment, the pre-formed polymer is in an amount of about 20% to 40%; the plasticizer is in an amount of about 15% to 35%; the wetting agent is in an amount of about 20% to 40%; the ion-balance reagent is in an amount of about 1% to 30%; the competitive reagent is in an amount of about 0.2% to 8%; and the indicator agent is in an amount of about 0.1% to 2%.

According to yet another embodiment, the pre-formed polymer is in an amount of about 25% to 37%; the plasticizer is in an amount of about 18% to 30%; the wetting agent is in an amount of about 22% to 37%; the ion-balance reagent is in an amount of about 1.5% to 28%; the competitive reagent is in an amount of about 0.4% to 5%; and the indicator agent is in an amount of about 0.3% to 1%.

According to a currently preferred embodiment, the pre-formed polymer is cellulose acetate; the plasticizer is dibutylphthalate or dioctylphthalate; the wetting agent is 2-ethoxy ethanol; the ion-balance reagent is methyl trioctyl-ammonium chloride or tri-dodecylmethyl ammonium chloride; the competitive reagent is selected from citric acid and tartaric acid and the indicator reagent is nitrazine yellow.

According to yet another embodiment, the composition further comprises a solvent. According to yet another embodiment, the solvent is selected from the group consisting of acetone, alcohol, diluted alcohol, amylene hydrate, benzyl benzoate, butyl alcohol, carbon tetrachloride, chloroform, corn oil, cottonseed oil, ethyl acetate, glycerin, hexylene glycol, isopropyl alcohol, methyl alcohol, methylene chloride, methyl isobutyl ketone, mineral oil, peanut oil, polyethylene glycol, propylene carbonate, propylene glycol, volatile ethers, tetrahydrofuran, sesame oil and water. According to a currently preferred embodiment the solvent is acetone.

According to another aspect, the present invention provides a bodily fluid-testing article comprising a substrate, an absorbent material, for absorbing the bodily fluid, wherein the substrate comprises the composition of the invention.

According to one embodiment, the article further comprises mounting means for placing the absorbent material in a position to receive the bodily fluid secreted from a person.

According to another embodiment, the absorbent material is selected from the group consisting of swab, gauze, panty shield, hygienic napkin, a diaper and interlabial absorbent structure.

According to yet another embodiment, the substrate is selected from the group consisting of polyester membranes, polypropylene membranes, cellulose membranes, paper, cotton and linen.

According to yet another embodiment, the composition is applied to said substrate by a method selected from the group consisting of dipping said substrate in said composition, spraying said composition on said substrate and spreading said composition over said substrate.

The present invention further provides a method for determining a medical condition of a subject comprising the steps of:
(a) providing an article comprising a composition for determining the presence of a charged analyte of interest in a tested bodily fluid, comprising a pre-formed polymer, a plasticizer, a wetting agent, an indicator reagent being charged oppositely to an analyte of interest in a tested bodily fluid, a competitive reagent having the same charge as the indicator reagent, and an ion-balance reagent, wherein the binding affinity of the competitive reagent to the analyte is stronger than the binding affinity of the indicator reagent to said analyte, and wherein the concentration of the competitive reagent determines a pre-set threshold of a visible indication such that upon contact of the composition with a bodily fluid comprising said analyte in a concentration above the pre-set threshold, said composition changes color;
(b) providing a color-encoding chart comprising a plurality of color codes and a description of medical condition for each color code;
(c) contacting the composition with tested bodily fluid;
(d) removing said composition from the tested bodily fluid; and
(e) comparing the color of said composition to the color-encoding chart and interpreting thereby determining the medical condition..

According to one embodiment, the color encoding chart specifies the color codes for a medical condition selected from vaginal infections, bacterial vaginosis, parasitic vaginosis and amniotic fluids. According to another embodiment, the color of the composition of step (a) does not change upon contact with urine.

According to yet another embodiment, the tested bodily fluid is selected from the group consisting of vaginal secretion, blood, saliva, ocular lens fluid, sweat, urine, milk, ascites fluid, mucous, synovial fluid, peritoneal fluid and amniotic fluid.

According to yet another embodiment, the ion in the tested bodily fluid comprises a quaternary amine. According to another embodiment, the quaternary amines is selected from the group consisting of trimethyl amine, ammonia, 1,5-pentane diamine, 1,4-butane diamine, spermine, spermidine and tyramine.

### DETAILED DESCRIPTION OF THE PREFERRED EMBODIMENTS

The present invention provides a bodily fluid-testing composition for the identification of a specific ion concentration exceeding a pre-set threshold in a tested bodily fluid, in which an ion oppositely charged to the ion in the tested bodily fluid is used to compete with an indicator reagent in order to compensate for variability in specific binding of the bodily fluid components. The compositions of the present invention should be capable of determining substantially different pH ranges, buffer capacities, and ion concentrations capable of reacting differently to different bodily fluids to produce a different color change.

Definitions

The terms “analyte” and “ion of interest” are interchangeably used herein to describe a charged compounds, the presence of which is indicative of a medical condition. According to currently particular embodiment, the bodily fluid is vaginal or amniotic fluid and the analyte of interest is a quaternary amine ion.

The term “specific binding”, as used herein, refers to binding of two different molecules wherein one of the molecules through chemical or physical means specifically binds to the second molecule, such as binding of an anion to a cation.

The term “tested bodily fluids”, as used herein, refers to virtually any bodily liquid sample. The test sample can be derived from any desired source, for example, vaginal secretion, blood, saliva, ocular lens fluid, sweat, urine, milk, ascites fluid, mucous, synovial fluid, peritoneal fluid, amniotic fluid or the like.

The term “indicator reagent”, as used herein, refers to a charged binding reagent, which produces a detectable signal upon contact with a charged analyte of interest in a tested bodily fluid. The magnitude and stability of the signal is commonly affected by environmental conditions, particularly, humidity, buffer capacity and pH of the tested bodily fluid. However, due to the unique content of the composition of the invention the indication produced by the indicator reagent is not affected by changes in pH on drying, interfering biological fluids and repetitive cycles of drying/wetting.

The term “competitive reagent”, as used herein, refers to a specific binding reagent, being charged which competes with an indicator reagent on the binding of an ion in a tested bodily fluid.

The term “pre-set threshold”, as used herein, refers to a specific threshold of ion concentration in a tested bodily fluid, for example ammonium concentration levels in tested urine at values lower or higher than 60 mM; biogenics amines such as diamines, trimethylamine, spermine and tyramine in tested secretion spanning from 0.01 mM to 50 mM; uric acid in tested urine from 0.1 mM to 50 mM, and 0.01 to 80 mM respectively.

The term “substantially different pH ranges” is to be construed in its most general sense and refers to any pH ranges that do not span exactly the same range. Namely, pH ranges having different upper limits and/or different lower limits are substantially different. These different pH ranges may comprise overlapping pH values, such as a pH range of 5.0-8.0 and a pH range of 4.0-7.0 and may be also essentially different, namely, devoid of any overlapping pH values.

The term “about” as used herein refers to +/−10%.

The terms “stable indication” and “irreversible indication” are interchangeably used herein to describe an indication, typically a color change that once obtained remains sufficiently altered for a time sufficient for clinical examination by a professional. Preferably the color change is stable for at least 48 hours, more preferably at least 72 hours, and in some embodiments, preferably the color change is stable for about a week.

Preferred Modes for Carrying Out the Invention

Accurate, absolute and fast determination of a medical condition is extremely important in various medical conditions, including, vaginal infections and amniotic leakage. As detailed above, a number of compositions and devices comprising same, having indicators for indicating medical conditions are known in the art. However, they often provide “false positives” due to changes in pH on drying, interfering biological fluids and repetitive cycles of drying/wetting. Vaginal infections and amniotic leakage are particular medical conditions that can be diagnosed using the articles known in the art, however, there are often misdiagnosed due to the plurality of substances having similar pH levels, which are commonly present in vaginal secretions. Inaccurate diagnosis of vaginal infections and amniotic leakage due to “false positive” readings is stressful and time consuming to the user.

Commonly, false positive readings of vaginal secretions are caused due to the presence of urine. The pH of vaginal secretions of a patient having bacterial vaginosis is between 4.7 and 6.5. The pH of urine of a healthy patient is within the range of 5.0 and 8.0. Thus, diagnosing bacterial vaginosis with a high degree of confidence cannot be achieved merely by pH-based test, unless the sample fluid is collected directly from the vagina, where urine is not ordinarily found. However, such examination is uncomfortable and requires intervention of a health-care professional.

According to one embodiment, the compositions of the present invention can be used for the identification of vaginal infections such as bacterial vaginosis (BV) or parasite. Bacterial vaginosis (BV) is characterized by production of increased quantities of malodorous vaginal discharge. The vaginal discharge of women with BV is described as being thin (low viscosity), off-white-gray (milk-like consistency), and homogeneous (distinctly not curd-like).

In the vagina there are no glands so that the fluid which it contains results from cervical secretion, vulvar secretions from sebaceous, sweat, Bartholine and Skeens glands, exfoliated cells, endometrial and oviductal fluids but mainly from liquid transudation through the vaginal epithelial walls.

As mentioned above, one of the characteristics of BV is the homogeneous discharge. A women having BV typically has an increase in the discharge amount. The source of this liquid is extracellular fluid (interstitial fluid) that surrounds the epithelial cells in the vagina wall. The ionic composition of the extracellular fluid and the plasma is quite similar with some differences reflecting the inability of large solutes, like proteins, to cross the cells wall.

A decrease in protein levels and other large organic molecules and the increase of water content in BV secretions lowers the buffering capacity of the secretions. Thus, secretions associated with BV have a lower buffer capacity than healthy vaginal secretions.

The composition of the invention is capable of providing an accurate determination of a medical condition due to its unique content, namely, a pre-formed polymer, an indicator reagent being charged oppositely to an analyte of interest in a tested bodily fluid, a competitive reagent having the same charge as the indicator reagent, and an ion-balance reagent, wherein the binding affinity of the competitive reagent to the analyte is stronger than the binding affinity of the indicator reagent to said analyte, and wherein the concentration of the competitive reagent determines a pre-set threshold of a visible indication such that upon contact of the composition with a bodily fluid comprising said analyte in a concentration above the pre-set threshold, said composition changes color. Optionally, the composition further comprises a plasticizer and/or a wetting agent.

According to one embodiment, the composition is hydrophobic thereby providing an indication of physiological conditions associated with the pH and/or the buffer capacities of the tested bodily fluid.

According to a currently preferred embodiment of the present invention, an indicating composition is made with nitrazine yellow that indicates the presence of a fluid with a pH of around 4.2 to 7.0. Upon contacting vaginal secretions having a pH of 5.2 or greater, the color changes from pale yellow to blue or green, which indicates possible BV or Trichomonas. At pH of 5.1 or lower, but greater than 4.2, the color change depends on the ionic strength of the vaginal discharge: the more fluidic is the discharge; the change in color is less evident. Fluids with pH levels of 4.2 or lower do not cause a change in the color of the indicating composition.

According to one preferred embodiment, the indicating composition comprises nitrazine yellow, which has a pKa of 6.6 in aqueous solution, and upon contacting vaginal secretions changes color, wherein without wishing to be bound to theory, the change in color results from the presence of organic acid such as acetic acid, citric acid and lactic acid in the vaginal secretion with pH levels of at least 5.0, without giving a false positive result due to urine interference.

According to another preferred embodiment, the minimal concentration of said organic acid is about 0.25 mM.

According to another embodiment of the present invention, the preformed can be selected from various preformed polymers such as cellulose, cellulose acetate, sodium carboxymethyl cellulose, ethyl cellulose, and nitrocellulose, although cellulose acetate is currently preferred.

The preformed polymer makes up 20% to 40% of the weight of the composition, wherein the percents are weight percent based on the total dry weight of the composition and the total dry weight of the composition equals 100%.

In certain embodiments, the polymer makes up 25% to 37% of the composition. As is clear to one skilled in the art, it is also possible to use a combination of suitable preformed polymers when making one polymer solution.

According to another embodiment of the present invention, a plasticizer can be selected from various plasticizers such as dibutylphthalate (DBP, CAS 84-74-2), dioctylphthalate, castor oil, diacetylated monoglycerides, diethyl phthalate (DEP, CAS 84-66-2), glycerin, mono- and di-acetylated monoglycerides, polyethylene glycol, propylene glycol, triacetin, triethyl citrate, bis-(2-butoxyethyl) adipate (BBPA, CAS 141-18-4), and bis-(2-ethylhexyl) sebacate (DOS, CAS 122-62-3), although dibutylphthalate and dioctylphthalate are currently preferred.

The plasticizer makes up 15% to 35%, 18% to 30%, 19% to 27%, by weight of the composition. As is clear to one skilled in the art, it is also possible to use a combination of suitable plasticizers when making one polymer solution.

According to a further embodiment of the present invention, an ion-balance reagent can be selected from various ion-balance reagents such as tri-dodecylmethyl ammonium chloride (TDMAC; CAS 7173-54-8), methyl trioctyl-ammonium chloride (Aliquat 336; CAS 5137-55-3), di(long-chain alkyl)dimethyl ammonium chloride, N-methyl-N,N-bis(long-chain alkanoyl oxyethyl)-N-(2-hydroxyethyl) ammonium methylsulfate, vinylbenzyl dimethylcocoammonium chloride, and cetyltimethyl ammonium chloride (CTAC; CAS 112-02-7), although tri-dodecylmethyl ammonium chloride and methyl trioctyl-ammonium chloride are currently preferred.

The ion-balance reagent makes up 1% to 30%, 1% to 29% and 1.5% to 28% by weight of the composition. As is clear to one skilled in the art, it is also possible to use a combination of suitable ion-balance reagents when making one polymer solution.

According to still another embodiment of the present invention, a wetting agent can be selected from various wetting agents such as 2-ethoxy ethanol, triethylene glycol, ethylene glycol, and sorbitol, although 2-ethoxy ethanol is currently preferred.

The wetting agent makes up 20% to 40%, 22% to 37% and 24% to 37% by weight of the composition. As is clear to one skilled in the art, it is also possible to use a combination of suitable wetting agents when making one polymer solution.

According to still a further embodiment of the present invention, a competitive reagent can be selected from various competitive reagents such as citric acid, oxalic acid, tartaric acid, succinic acid, glutaric acid, lactic acid, pyruvic acid, hydroxypropionic acid, hydroxyvaleric acid, adipic acid, suberic acid, orotic acid, phthalic acid, 2-[(2-hydroxy-1,1-bis(hydroxymethyl)ethyl)amino]ethanesulfonic acid and 4-(2-hydroxyethyl)piperazine-1-ethanesulfonic acid, although citric acid and tartaric acid are currently preferred.

The competitive reagent makes up 0.2% to 8% and 0.4% to 5% by weight of the composition.

Having now generally described the invention, the same will be more readily understood through reference to the following examples, which are provided by way of illustration and are not intended to be limiting of the present invention.

A second example of a medical condition that can be diagnosed by an accurate analysis of vaginal secretions is amniotic fluid leaking during pregnancy. Such leakage occurs when the amniotic sac integrity is compromised. If diagnosed as such, measures such as rest or sealing of the amniotic sac may be prescribed. If misdiagnosed, the amniotic sac may later rupture causing abortion of the pregnancy, extended hospitalization or premature birth.

Due to the severe consequences of amniotic fluid leakage, pregnant women undergo severe stress and often go to a health-care professional upon secretion of any liquid from the vicinity of the vagina. The health-care professional looks for the presence of amniotic fluid by checking the pH of the vaginal secretions, amniotic fluid having a pH of between 6.0 and 8.0. Since pregnant women often have urinary incontinence and since urine typically has a pH of between 5.0 and 8.0, if only pH is checked, a false positive result may occur: urine being identified as amniotic fluid. Consequently, it is necessary that such a vaginal secretion be examined using a microscope for the presence of a fern-shaped pattern indicative of amniotic fluid.

As the time between the fluid secretion and the arrival at the health-care professional may be long, there is often no evidence of amniotic fluid upon examination. The secretion may mistakenly be assumed to be urine, often with tragic consequences. On the other hand, the healthcare professional may decide to err on the side of caution, misdiagnosing the secretion of urine as amniotic fluid leading to an unnecessary hospitalization and patient stress.

Thus, the compositions and articles of the invention are particularly useful for detecting amniotic leakage as they enable to distinguish accurately between normal and abnormal ammonium concentrations in urine, to thereby indicate the hydration level of monitored subject. According to one embodiment, the article of the present invention can be used for the identification of amniotic fluid leaking from the vagina of a pregnant woman. The article of the present invention is able to detect pH changes above a pre-set threshold specific to amniotic fluid in the vaginal fluids.

According to some embodiments, the compositions of the invention enable to distinguish accurately between ammonium concentrations levels at values lower or higher than 60 mM.

Yet another medical condition requiring an accurate determination of specific ion concentration in a tested bodily fluid is dehydration. Dehydration is a condition in which the body or certain body tissues suffer from lack of water and important blood ions like potassium (K+) and sodium (Na+). Vital organs like the kidneys, brain, and heart cannot function without a certain minimum of water and salt. Tissue dehydration may occur in dry climates and during the winter heating season. Extremely dry air causes the rapid evaporation of water from the skin and from the mucous linings of the respiratory system.

Causes of dehydration include excessive fluid losses, inadequate fluid intake, or a combination of these factors. Illnesses that produce diarrhea and vomiting are common causes of dehydration, since both conditions cause loss of body fluids. Other causes of dehydration include diabetes, kidney disease, excessive use of diuretics, liver disease resulting in accumulation of fluid in the abdominal cavity, inflammation of the abdominal cavity resulting in fluid accumulation and burns.

Premature or preterm babies, infants and children are more susceptible to dehydration than adults because of their smaller body weights and higher turnover of water and electrolytes. The elderly and those with illnesses are also at higher risk. In underdeveloped countries, dehydration from diseases like cholera and dysentery kills millions every year (usually infants and children).

For infants, dehydration can develop quickly and even become life threatening if not treated properly. It is therefore very important to recognize the dehydration instantly. The early symptoms of dehydration are urinating smaller amounts than usual and dark yellow urine, since the kidneys retain more water and urine is more concentrated. The color and clarity of urine, the urine specific gravity, and the presence of ketones in the urine may all help to indicate the degree of dehydration. A high urine specific gravity indicates significant dehydration.

Thus, according to one embodiment, the article of the present invention can be used to distinguish accurately between normal and abnormal ammonium concentration in the tested urine without any interference of other biological fluids. The article of the present invention is able to detect ammonium cations below or above a pre-set threshold of 60 mM. The present invention provides a monitoring article that uses ammonium ions concentration in the urine as an indicator of the hydration state of the tested subject.

Dehydration is classified as mild, moderate, or severe based on how much of the body's fluid is lost or not replenished, depending on age. Mild dehydration is defined as a loss of 3-5% of body weight; Moderate dehydration is defined as a loss of 6-10% of body weight; and severe dehydration is defined as a loss of more than 9-15% of body weight. When severe, dehydration is a life-threatening emergency.

The present invention provides a bodily fluid-testing article comprising the compositions of the invention. The article for monitoring of bodily fluids comprises a substrate and an absorbent material for absorbing said bodily fluid, the substrate comprising a composition suitable for identification of a specific ion concentration exceeding a pre-set threshold in a tested bodily fluid, particularly, amniotic and vaginal fluids.

The article can be embodied as a swab, gauze, shield, hygienic napkin, diaper or interlabial absorbent structure and can be used to indicate the presence of abnormal ammonium concentration in human urine, amniotic fluid leakage, or biogenic secretions associated with bacterial vaginosis, parasite infections, or deficiency of lactobacillus population, without giving a false positive result.

Advantageously, the article is well suited for all types of use, for example in pediatrics, geriatrics, and gynecology.

The bodily fluid-testing article can be implemented using many devices and methods. In a preferred embodiment, the article of the present invention is implemented in a manner that can be easily used by non-skilled personnel, specifically a user. The substrate of the article of the present invention comprising the absorbent material can be supplied to the user, for example, in the form of a pad, gauze, a swab, a fiber ball, but most preferably, as a sanitary napkin, diaper, panty shield, and interlabial structure. Details of manufacture of these are well known to one skilled and have been fully described in the prior art, for example U.S. Pat. Nos. 5,217,444, 5,897,834, and 6,149,590.

Furthermore, any user, male or female, young or old, can use the article in a variety of forms. The particular examples of the invention as presented herein are not intended to limit the scope of the invention, but simply to illustrate and represent the numerous potential forms in which the invention can be used.

In other embodiments of the bodily fluid-testing article, a means for mounting the article to facilitate the collection of the bodily fluid is included. An example of a mounting means that is well known in the art is an adhesive strips associated with the article. In a preferred embodiment the article has one or more adhesive strips. The user removes the release tape to expose the adhesive strip of the article and places the article in the crotch portion of their undergarment. This prevents the article from moving out of position during regular use. Types of adhesive compounds that can be used are well known in the art.

The article can be configured to identify abnormal ammonium concentrations in urine, amniotic fluid and vaginal secretions associated with bacterial, parasite, fungal, or yeast infection. Furthermore, the article is designed to minimize false positive readings associated with interfering biological fluids.

When used in a medical setting, it is imperative that there be substantially no leaching of the composition components from the substrate to which the composition is attached. The attachment of composition to a substrate is well within the ability of one skilled in the art. Chemical compounds suitable for use as the indicators of the present invention without leaching are indicators with negative functional groups. Suitable indicators include nitrazine yellow, thymol blue, bromthymol blue, xylenol blue, bromoxylenol blue, phenol red, m-cresol purple, chlorophenol red, bromcresol purple, alizarin, neutral red, and cresol red. A list of other suitable indicators can be found, for example, in U.S. Pat. No. 5,897,834. It is clear to one skilled in the art that the indicators specifically mentioned herein are just examples and any suitable indicators may be used.

The present invention further provides a method for determining a medical condition of a subject comprising the steps of:
(a) providing an article comprising a composition for determining the presence of a charged analyte of interest in a tested bodily fluid, comprising a pre-formed polymer, a plasticizer, a wetting agent, an indicator reagent being charged oppositely to an analyte of interest in a tested bodily fluid, a competitive reagent having the same charge as the indicator reagent, and an ion-balance reagent, wherein the binding affinity of the competitive reagent to the analyte is stronger than the binding affinity of the indicator reagent to said analyte, and wherein the concentration of the competitive reagent determines a pre-set threshold of a visible indication such that upon contact of the composition with a bodily fluid comprising said analyte in a concentration above the pre-set threshold, said composition changes color;
(b) providing a color-encoding chart comprising a plurality of color codes and a description of medical condition for each color code;
(c) contacting the composition with tested bodily fluid;
(d) removing said composition from the tested bodily fluid; and
(e) comparing the color of said composition to the color-encoding chart and interpreting thereby determining the medical condition.

According to one embodiment, the color encoding chart specifies the color codes for a medical condition selected from vaginal infections, bacterial vaginosis, parasitic vaginosis and amniotic fluids. According to another embodiment, the color of the composition of step (a) does not change upon contact with urine.

According to yet another embodiment, the tested bodily fluid is selected from the group consisting of vaginal secretion, blood, saliva, ocular lens fluid, sweat, urine, milk, ascites fluid, mucous, synovial fluid, peritoneal fluid and amniotic fluid.

According to yet another embodiment, the ion in the tested bodily fluid comprises a quaternary amine. According to another embodiment, the quaternary amines is selected from the group consisting of trimethyl amine, ammonia, 1,5-pentane diamine, 1,4-butane diamine, spermine, spermidine and tyramine.

### EXAMPLES

### Example 1

### Reducing Erroneous Readings of Color-Changing Articles by Using a Charged Competitor

The following example provides guidelines for the production of an article, which can preferentially detect positively charged compounds, such as quaternary amines, in a tested bodily fluid such as vaginal secretion or amniotic fluid. Using a negatively charged competitor, such as an organic acid, having a greater binding affinity to interfering cations in a tested bodily fluid than the affinity of a negatively charged indicator reagent, such as nitrazine yellow, results in an accurate indication of the presence of an ion of interest in the tested bodily fluid and enables to avoid false positive readings due to urine contamination.

An indicator reagent produces a color, or induces a color change, when the amount of bonded cations per surface is large enough. The end point of the reaction between the tested bodily fluid and the indicator reagent is monitored by the concentration of the competing organic acid, which should preferentially bind to the free cations. The following two equations demonstrate the competitive reactions.
RCOOH+NH₄⁺+OH⁻→RCOONH₄+OH⁻+H⁺
InH+X⁻+Y⁺→InY+X⁻+H⁺
KEY:
RCOOH=Organic Acid
InH=Indicator reagent
X⁻=OH⁻
Y⁺=Quaternary amines
If [RCOOH]≧[Y⁺] no color change will take place. If [RCOOH]<[Y⁺] a color change will occur.

Providing physicians with a reliable clinic instantaneous detecting article, that distinguishes vaginal secretions or amniotic fluid leakage from urine incontinence with no false alarms, can serve them by far better than available solutions today.

### Example 2

### Detection of the Concentration of Ammonium Ions in Urine Exceeding a Pre-set Threshold

The following example discloses a solution to produce an article, which can distinguish accurately between normal and abnormal ammonium concentration in tested urine by a visual color change, without any interference of other biological fluids. The example provides a diagnostic article for the detection of dehydration by using a negatively charged competitor such as an organic acid, with a greater binding affinity to the detected ammonium cations in the tested urine than the negatively charged indicator reagent such as nitrazine yellow.

The indicator reagent shows a color change, when the amount of bonded ammonium cations per surface is large enough. The end point of the reaction between the tested urine and the indicator reagent is determined by the amount of left bonded ammonium cations per surface after the reaction is finished, including a dry-out phase. The threshold of the indication of the ammonium concentration is controlled by the ratio between the organic acid and indicator concentrations. As the organic acid concentration is increased the sampled urine needs a greater concentration of ammonium cations, to fully reverse the color changes. Observation of a visible stable color at the end of the reaction indicates that the ammonium concentration is greater than the pre-set threshold. The following two equations demonstrate the competitive reactions.
RCOOH+NH₄⁺+OH⁻→RCOONH₄+H₂O
InH+NH₄⁺+OH⁻→InNH₄+H₂O
KEY:
RCOOH=Organic Acid
InH=Indicator reagent
NH₄⁺=ammonium cation

Generally, urine with lower pH such as 5-5.5 and with normal ammonium cation concentration (30-50 mM) fully reverses the change in the indicator color. On the contrary, a reaction of an indicator with urine at pH 5-8 and ammonium cation concentration above 60 mM changes the color of the indicator to a stable color.

### Example 3

### A Composition for the Identification of Vaginal Secretions Associated with Bacterial Vaginosis

The composition for the identification of vaginal secretions associated with bacterial vaginosis containing biogenic amines, such as trimethylamine, 1,4-diaminobutane and 1,5-diamino pentane, comprising cellulose acetate in an amount of 34.6%; dibutylphthalate in an amount of 25.8%; 2-ethoxy ethanol in an amount of 32.2%; tri-dodecylmethyl ammonium chloride (Aliquat 336) in an amount of 4.7%; nitrazine yellow in an amount of 0.6%; and citric acid in an amount of 2.2%; wherein the percents are weight percent based on the total dry weight of the composition and the total dry weight of the composition is 100% (Table 1).

TABLE 1
Components of the vaginal secretions indicating composition

| Function | % W/W | Chemical |
|---|---|---|
| Polymer | 34.60 | Cellulose Acetate |
| Plasticizer | 25.80 | Dibutylphthalate |
| Ion-balance reagent | 4.70 | Aliquat 336 |
| Wetting agent | 32.20 | 2-Ethoxy Ethanol |
| Indicator | 0.60 | Nitrazine Yellow |
| Competitive reagent | 2.20 | Citric Acid |

The method for preparation of an article for the identification of vaginal secretions associated with bacterial vaginosis comprising the steps of:
Step 1: To 85.98 ml of acetone add 3.61 g cellulose acetate, 2.58 ml dibutylphthalate, 0.55 ml Aliquat, 3.61 ml 2-Ethoxy ethanol, 0.46 g citric acid and 0.06 g nitrazine yellow dissolved in 3.61 ml DDW.
Step 2: Stir the mixture for few minutes to complete dissolving.
Step 3: Coat a polyester non-woven fabric with the polymer solution to yield the desired product.
Step 4: Dry over night.

### Example 4

### A Composition for the Detection of Amniotic Fluid without Urine Interference

The composition for the detection of amniotic fluid without urine interference comprising cellulose acetate in an amount of 34.2%; dibutylphthalate in an amount of 25.5%; 2-ethoxy ethanol in an amount of 31.8%; Aliquat 336 in an amount of 4.6%; nitrazine yellow in an amount of 0.5%; and tartaric acid in an amount of 3.4%; wherein the percents are weight percent based on the total dry weight of the composition and the total dry weight of the composition equals 100% (Table 2).

TABLE 2
Components of the amniotic fluid detecting composition

| Function | % W/W | Chemical |
|---|---|---|
| Polymer | 34.20 | Cellulose Acetate |
| Plasticizer | 25.50 | Dibutylphthalate |
| Ion-balance reagent | 4.60 | Aliquat 336 |
| Wetting agent | 31.80 | 2-Ethoxy Ethanol |
| Indicator | 0.50 | Nitrazine Yellow |
| Competitive reagent | 3.40 | Tartaric Acid |

The method for preparation of an article for the identification of amniotic fluid without urine interference comprising the steps of:
Step 1: To 89.6 ml of acetone add 2.7 g cellulose acetate, 1.9 ml dibutylphthalate, 0.4 ml Aliquat 336, 2.7 ml 2-ethoxy ethanol, 0.4 g tartaric acid and 0.04 g nitrazine yellow dissolved in 2.7 ml DDW.
Step 2: Stir the mixture for few minutes to complete dissolving.
Step 3: Coat a polyester non-woven fabric with the polymer solution to yield the desired product.
Step 4: Dry over night.

### Example 5

### Method for Preparation of Vaginal Secretion-monitoring Swab

The composition for the preparation of vaginal secretion monitoring swab for the detection of biogenic amines such as trimethylamine, 1,4-diamino butane and 1,5-diamino pentane, comprising cellulose acetate in an amount of 36.2%; dioctylphthalate in an amount of 25.4%; 2-ethoxy ethanol in an amount of 33.7%; tri-dodecylmethyl ammonium chloride (TDMAC) in an amount of 2.4%; nitrazine yellow in an amount of 0.6%; and citric acid in an amount of 1.6%; wherein the percents are weight percent based on the total dry weight of the composition and the total dry weight of the composition equals 100% (Table 3).

TABLE 3
Components of the vaginal secretion-monitoring swab

| Function | % W/W | Chemical |
|---|---|---|
| Polymer | 36.26 | Cellulose Acetate |
| Plasticizer | 25.39 | Dioctylphthalate |
| Ion-balance reagent | 2.41 | TDMAC |
| Wetting agent | 33.73 | 2-Ethoxy Ethanol |
| Indicator | 0.58 | Nitrazine Yellow |
| Competitive reagent | 1.63 | Citric Acid |

The method for the preparation of vaginal secretion monitoring swab comprising the steps of:
Step 1: To 80.5 ml of acetone add 0.8 g cellulose acetate, 0.6 ml dioctylphthalate, 0.05 ml tri-dodecylmethyl ammonium chloride (TDMAC), 0.8 ml 2-ethoxy ethanol, 0.9 g citric acid and 0.01 g nitrazine yellow dissolved in 0.8 ml DDW.
Step 2: Stir the mixture for few minutes to complete dissolving.
Step 3: Coat a swab with tip made of polyester fabric with the polymer solution to yield the desired product.
Step 4: Dry over night.

The tip may be prepared by using a short strip, rolled on the stick of the swab, or by coating the tip of an integrated swab, where the tip consists of any screening fabric.

The composition is applied to the swab for example by dipping the swab in the composition or by spraying or spreading the composition onto the swab. The swab with the applied composition is allowed to dry. When dry, the indicator is bound to the substrate with the help of the polymer.

### Example 6

### A Composition for the Detection of Elevated pH Values in Vaginal Secretions without Urine Interference

The composition for the detection of elevated pH values in vaginal secretions containing organic acids such as acetic acid, citric acid and lactic acid, at minimal concentrations, wherein the pH level spans from 4.5 to 7.0 for acetic acid at concentration threshold of 7.6 μg/ml or higher, and pH 6.0 for lactic acid at concentration threshold of 6.6μg/ml or higher, (the same innovative specific composition produces indicators for various organic acids), indistinguishably of urine remains.

The composition comprises cellulose acetate in an amount of 27.3%; dioctylphthalate in an amount of 19.1%; 2-ethoxy ethanol in an amount of 25.39%; tri-dodecylmethyl ammonium chloride (TDMAC) in an amount of 27.3%; nitrazine yellow in an amount of 0.48%; and citric acid in an amount of 0.43%; wherein the percents are weight percent based on the total dry weight of the composition and the total dry weight of the composition equals 100% (Table 4).

TABLE 4
Components of the vaginal secretion detecting composition

| Function | % W/W | Chemical |
|---|---|---|
| Polymer | 27.30 | Cellulose Acetate |
| Plasticizer | 19.10 | Dioctylphthalate |
| Ion-balance reagent | 27.30 | TDMAC |
| Wetting agent | 25.39 | 2-Ethoxy Ethanol |
| Indicator | 0.48 | Nitrazine Yellow |
| Competitive reagent | 0.43 | Citric Acid |

The method for preparation of an article for the identification of vaginal secretions, containing organic acids with a pH level of 5.0 or higher comprising the steps of:
Step 1: To 10 ml of acetone add 0.31 g cellulose acetate, 0.22 ml dioctylphthalate, 0.31 ml 2-ethoxyethanol, 0.02 g tri-dodecylmethyl ammonium chloride (TDMAC), 0.005 g citric acid, and 0.006 mg nitrazine yellow dissolved in 0.3 1 ml DDW.
Step 2: Stir the mixture for few minutes to complete dissolving.
Step 3: Coat the polyester non-woven fabric with the polymer solution to yield the desired product.
Step 4: Dry over night.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

What is claimed is

1. A composition for determining the presence of a charged analyte of interest in a tested bodily fluid, comprising a pre-formed polymer, an indicator reagent being charged oppositely to an analyte of interest in a tested bodily fluid, a competitive reagent having the same charge as the indicator reagent, and an ion-balance reagent, wherein the binding affinity of the competitive reagent to the analyte is stronger than the binding affinity of the indicator reagent to said analyte, and wherein the concentration of the competitive reagent determines a pre-set threshold of a visible indication such that upon contact of the composition with a bodily fluid comprising said analyte in a concentration above the pre-set threshold, said composition changes color.

2. The composition of claim 1, wherein the indicator reagent and the competitive reagent are negatively charged.

3. The composition of claim 1, wherein the indicator reagent and the competitive reagent are positively charged.

4. The composition of claim 1, wherein the indicator reagent is a weak organic acid and the competitive reagent is an organic acid and the competitive reagent is an organic acid.

5. The composition of claim 1, wherein the indicator reagent is a weak organic base and the competitive reagent is an organic base.

6. The composition of claim 1, wherein the indicator reagent is selected from the group consisting of methyl yellow, methyl orange, bromophenol blue, alizarin sodium sulfonate, naphtyl red, bromcresol green, methyl red, bromcresol purple, nitrazine yellow, bromoxylenol blue, neutral red, phenol red, thymol blue, xylenol blue, m-cresol purple, naphtholthalein, phenolphthalein and naphtholbenzein.

7. The composition of claim 1, wherein the competitive reagent is selected from the group consisting of citric acid, oxalic acid, tartaric acid, succinic acid, glutaric acid, lactic acid, pyruvic acid, hydroxypropionic acid, hydroxyvaleric acid, adipic acid, suberic acid, orotic acid, phthalic acid, 2-[(2-hydroxy-1,1-bis(hydroxymethyl)ethyl)amino]ethanesulfonic acid and 4-(2-hydroxyethyl)piperazine-1-ethanesulfonic acid.

8. The composition of claim 1, wherein the competitive reagent is selected from the group consisting of cyclodextrine sulfate, dextran sulfate, and carboxymethyl cellulose.

9. The composition of claim 1, wherein the ion-balance reagent is a quaternary amine selected from the group consisting of di(long-chain alkyl)dimethyl ammonium chloride, N-methyl-N,N-bis(long-chain alkanoyl oxyethyl)-N-(2-hydroxyethyl) ammonium methylsulfate, vinylbenzyl dimethylcocoammonium chloride, tri-dodecylmethyl ammonium chloride and methyl trioctyl ammonium chloride.

10. The composition of claim 1, wherein the pre-formed polymer is selected from the group consisting of cellulose acetate, cellulose, sodium carboxymethyl cellulose, ethyl cellulose and nitrocellulose.

11. The composition of claim 1, further comprises at least one compound selected from: a wetting agent and a plasticizer.

12. The composition of claim 11, wherein the wetting agent is selected from the group consisting of 2-ethoxy ethanol, triethylene glycol, ethylene glycol and sorbitol.

13. The composition of claim 11, wherein the plasticizer is selected from the group consisting of dibutylphthalate, dioctylphthalate, castor oil, diacetylated monoglycerides, diethyl phthalate, glycerin, mono- and di-acetylated monoglycerides, polyethylene glycol, propylene glycol, triacetin, triethyl citrate, bis-(2-butoxyethyl) adipate, and bis-(2-ethylhexyl) sebacate.

14. The composition of claim 1, wherein the pre-formed polymer is in an amount that does not exceed about 40%; the plasticizer is in an amount that does not exceed about 35%; the wetting agent is in an amount that does not exceed about 40%; the ion-balance reagent is in an amount that does not exceed about 30%; the competitive reagent is in an amount that does not exceed about 8% and the indicator reagent is in an amount that does not exceed about 2%; wherein the percents are weight percent based on the total dry weight of the composition and the total dry weight of the composition equals 100%.

15. The composition of claim 14, wherein the pre-formed polymer is cellulose acetate; the plasticizer is dibutylphthalate or dioctylphthalate; the wetting agent is 2-ethoxy ethanol; the ion-balance reagent is methyl trioctyl-ammonium chloride or tri-dodecylmethyl ammonium chloride; the competitive reagent is citric acid or tartaric acid; and the indicator reagent is nitrazine yellow.

16. The composition of claim 1, further comprising a solvent.

17. The composition of claim 16, wherein the solvent is selected from the group consisting of acetone, alcohol, diluted alcohol, amylene hydrate, benzyl benzoate, butyl alcohol, carbon tetrachloride, chloroform, corn oil, cottonseed oil, ethyl acetate, glycerin, hexylene glycol, isopropyl alcohol, methyl alcohol, methylene chloride, methyl isobutyl ketone, mineral oil, peanut oil, polyethylene glycol, propylene carbonate, propylene glycol, volatile ethers, tetrahydrofuran, sesame oil and water.

18. An article comprising a substrate and an absorbent material for absorbing bodily fluids, wherein the substrate comprises the composition of claim 1.

19. The article of claim 18, further comprising mounting means for placing the absorbent material in a position to receive bodily fluids secreted from a person.

20. The article of claim 18, wherein the absorbent material is selected from the group consisting of swab, gauze, panty shield, hygienic napkin, a diaper and interlabial absorbent structure.

21. The article of claim 18, wherein the substrate is selected from the group consisting of polyester membranes, polypropylene membranes, cellulose membranes, paper, cotton and linen.

22. A method for determining a medical condition of a subject comprising the steps of:
(a) providing an article comprising a composition for determining the presence of a charged analyte of interest in a tested bodily fluid, comprising a pre-formed polymer, a plasticizer, a wetting agent, an indicator reagent being charged oppositely to an analyte of interest in a tested bodily fluid, a competitive reagent having the same charge as the indicator reagent, and an ion-balance reagent, wherein the binding affinity of the competitive reagent to the analyte is stronger than the binding affinity of the indicator reagent to said analyte, and wherein the concentration of the competitive reagent determines a pre-set threshold of a visible indication such that upon contact of the composition with a bodily fluid comprising said analyte in a concentration above the pre-set threshold, said composition changes color;
(b) providing a color-encoding chart comprising a plurality of color codes and a description of medical condition for each color code;
(c) contacting the composition with tested bodily fluid;
(d) removing said composition from the tested bodily fluid; and
(e) comparing the color of said composition to the color-encoding chart and interpreting thereby determining the medical condition.

23. The method of claim 22, wherein the color-encoding chart specifies the color codes for a medical condition selected from vaginal infections, bacterial vaginosis, parasitic vaginosis and amniotic fluids.

24. The method of claim 22, wherein the tested bodily fluid is selected from the group consisting of vaginal secretion, blood, saliva, ocular lens fluid, sweat, urine, milk, ascites fluid, mucous, synovial fluid, peritoneal fluid and amniotic fluid.

25. The method of claim 22, wherein the ion in the tested bodily fluid comprises a quaternary amine.

26. The method of claim 25, wherein the quaternary amines is selected from the group consisting of trimethyl amine, ammonia, 1,5-pentane diamine, 1,4-butane diamine, spermine, spermidine and tyramine.
